(19) United States
(12) Patent Application Publication
(10) Pub. No.: US 2016/0223543 A1
(43) Pub. Date: Aug. 4, 2016

(54) SYSTEMS AND METHODS FOR TEMPERATURE CORRECTION IN TEST STRIPS FOR ENZYME DETECTION

(71) Applicant: Polymer Technology Systems, Inc., Indianapolis, IN (US)

(72) Inventor: Keith Moskowitz, Indianapolis, IN (US)

(21) Appl. No.: 15/012,348

(22) Filed: Feb. 1, 2016

Related U.S. Application Data
(60) Provisional application No. 62/110,015, filed on Jan. 30, 2015.

(51) Int. Cl. G01N 33/573; G01N 33/558

(52) U.S. Cl. CPC G01N 33/573; G01N 33/558; G01N 2333/805

(57) ABSTRACT

A system for detection of an analyte includes a lateral flow test strip, including a stripe for reacting with an analyte. The system further includes a meter including and executing instructions for reading the lateral flow test strip and determining a level of the analyte. In the system, the level of the analyte determined and adjusted according to an ambient temperature.

### CROSS-REFERENCES TO RELATED APPLICATIONS

This application claims the benefit of Provisional Application No. 62/100,015 filed on Jan. 30, 2015, titled “Systems And Methods For Temperature Correction In Test Strips For Enzyme Detection,” the entire disclosure of which is hereby incorporated by reference.

### BACKGROUND

Vertical flow and lateral flow test strips are unique and innovative mediums providing for point-of-care testing of blood analytes. While this technology can approach the accuracy provided by laboratory techniques, there are many peculiarities related to these test strips that make their operation, construction, use, and calibration difficult.

Temperature is one factor that can affect the accuracy of vertical and lateral flow test strips. Typically, vertical flow test strips include multiple layers that isolate portions of the sample so that they may be reacted and measured. In the case of analytes like cholesterol and the fractions of cholesterol, the layers may include a hematocrit separation layer, an analyte isolation layer which may isolate certain lipids from other lipids, and a reaction layer. The reaction layer typically relies on an enzyme that reacts with the total or isolated lipids in the sample. The reactivity and efficiency of enzymes at creating color from a sample may be affected by temperature.

Similarly, lateral flow test strips may isolate analytes from other parts of a sample by flowing the sample across a lateral medium. Typically, lateral flow test strips include stripes or reaction areas on the lateral flow test strip. These stripes change color in response to the presence of an analyte. These stripes include an antibody that reacts with the analyte to be tested for. Competitive type assays and sandwich type assays are common in lateral flow test strips. In more advanced lateral flow test strips, the color change of the stripe may be measured using a light source and an optical sensor. Lateral flow test strips are more often used to test for enzymes. As noted above, the activity of enzymes may be affected by temperature.

### BRIEF SUMMARY

In one embodiment, a system for detection of an analyte includes a lateral flow test strip, including a stripe for reacting with an analyte. The system further includes a meter including and executing instructions for reading the lateral flow test strip and determining a level of the analyte. In the system, the level of the analyte determined is adjusted according an ambient temperature. Optionally, the instructions include an algorithm for correlating a characteristic of the lateral flow test strip to the level of the analyte. Alternatively, the characteristic is color. Optionally, the meter includes a thermometer, the thermometer operable for detecting the ambient temperature. In one alternative, the analyte is an enzyme. In another alternative, the enzyme is A1C. Optionally, the algorithm is based on a calibration for 25° C., and the characteristic is adjusted based on the difference in the ambient temperature from the calibration. Alternatively, the stripe includes antibodies that react to the analyte.

In one embodiment, a chip for use with a lateral flow test strip, the lateral flow test strip including a stripe for reacting with an analyte, a meter including and executing instructions for reading the lateral flow test strip and determining a level of the analyte, includes a non-transitory computer medium readable by the meter, the non-transitory computer medium including instructions for determining the level of the analyte adjusted according to ambient temperature. Optionally, the instructions include an algorithm for correlating a characteristic of the lateral flow test strip to the level of the analyte. Alternatively, the characteristic is color. Optionally, the meter includes a thermometer, the thermometer operable for detecting the ambient temperature. In one alternative, the analyte is an enzyme.

In one embodiment, a method of determining an analyte level includes providing a lateral flow test strip, including a stripe for reacting with an analyte; and a meter including and executing instructions for reading the lateral flow test strip and determining a level of the analyte. The method further includes placing a sample on the lateral flow test strip and reading the lateral flow test strip with the meter. The method further includes adjusting the level of the analyte determined by the meter based on the ambient temperature. Optionally, the instructions include an algorithm for correlating a characteristic of the lateral flow test strip to the level of the analyte. Alternatively, the characteristic is color. In one alternative, the meter includes a thermometer, the thermometer operable for detecting the ambient temperature. Optionally, the analyte is an enzyme.

### BRIEF DESCRIPTION OF THE FIGURES

FIG. 1 shows one embodiment of a color adjustment algorithm based on the ambient temperature.

### DETAILED DESCRIPTION

Certain terminology is used herein for convenience only and is not to be taken as a limitation on the embodiments of the systems, compositions, and methods of temperature correction in test strips for enzyme detection.

In some embodiments, a lateral flow test strip is used in conjunction with a meter to test for the concentration of an analyte. In many embodiments, the analyte is an enzyme. Examples of enzymes include A1C (glycated hemoglobin) or other enzymes that can bind with antibodies. The antibodies may be located in a stripe on the lateral flow test strips. Either a competitive or sandwich type assay may be used.

An issue is that the enzyme activity at different temperatures may be lessened or increased. Therefore, the amount of color change may be increased or lessened depending on the type of assay used. This is because the enzyme may not react as quickly at lower temperatures. In the case of a lateral flow test strip, where a meter is used to read the strip and calculate a level of analyte in the sample, the meter may include a light source and a sensor. The sensor detects the reflectivity, color change, fluorescence, or other feature of the stripe. This detected level is converted via an algorithm in the meter to a level of analyte in the sample. In some configurations, the algorithm may be stored on an insertable chip or similar item. Therefore, particular test strips may be provided with particular chips including algorithms for the test strips.

Therefore, in some embodiments, the algorithm for calculating concentration of an analyte from the color change may be modified in relation to the temperature detected by the meter. To accommodate this change, the meter may include a temperature sensor to detect ambient temperature and provide the proper calibration to the meter.

In many embodiments, enzymatic activity doubles about every 10° C. That means that, under typical ambient conditions, there can be twice the amount of color produced during an end product measurement by the exact same amount of enzyme and thus can be reported relative between 1× and 2× the actual amount. Typical ambient conditions may range from 15° C. to 30° C.

Most analytical systems have temperature controls that heat the entire system to 37° C. (body temperature) to avoid issues with temperature. However, in the context of a lateral flow test strip such temperature controls may not be feasible or economical.

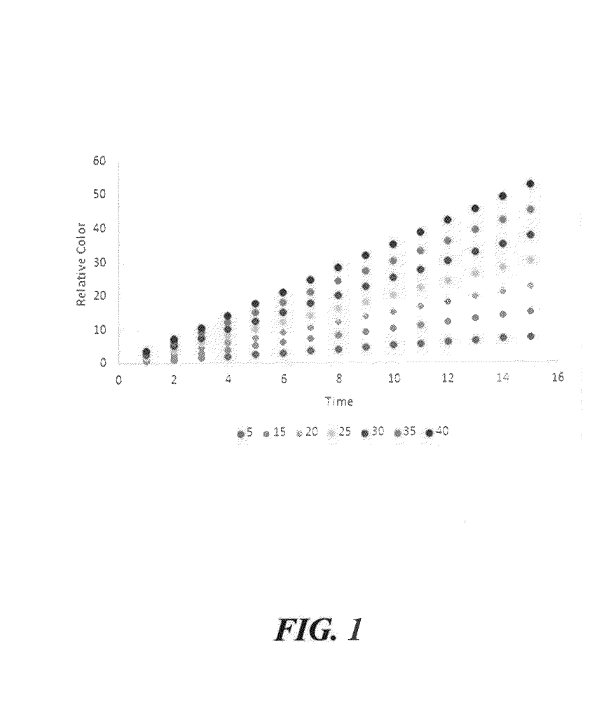

Accordingly, an algorithm based on the graph below may be used to adjust the relative color (fluorescence, reflectivity, or other feature) detected by the meter. FIG. 1 shows one embodiment of a color adjustment algorithm based on the ambient temperature. The Y-axis shows the relative color detected by the meter. The X-axis shows the time. The various color data points are representative of the anticipated difference in enzyme activity based on the temperature of the sample. As is visible in the FIGURE, the trend lines for the color change has a greater slope at higher temperatures and a lesser slope at lower temperatures.

Accordingly, the same amount of enzyme will produce these different colors based upon temperature.

The existing algorithm for the conversion of a measured color change to an amount of analyte in the sample may be modified according to the temperature. The algorithm assumes curve fitting to the reference analyzer at 25° C. (yellow), then use the temperature taken to adjust up or down based upon a comparison of slopes and/or the sin angle function for the algorithm. The actual algorithm for calculation of temperature adjustment may include results of enzyme activities at many additional levels or alternatively may interpolate between known levels. Activity trends are believed to be consistent across normally encountered temperature ranges.

In many embodiments, the correction algorithm is provided in the form of a chip, such as a MEMo Chip. The chip may include a bias function. The bias function may operate by storing an ambient temperature value at the moment the first test runs after the system is powered up. In some embodiments, the ambient temperature value is not updated until the next powerup. The chip may include a memory location programmed to the ambient temperature at the time the temperature curve was set in quality control operations.

As an example, the chip additionally may be programmed with a percent value. A value of 100% results in +/−1 mg/dL per Degree C of delta from the curve set temperature. A value of 50% results in +/−0.5 mg/dL per Degree C of delta from the curve set temperature. A value of 150% results in +/−1.5 mg/dL per Degree C of delta from the curve set temperature. The change in temperature between the startup temperature and the temperature at the end of the test is calculated using, for example, the equation:

CurveSetTemperature−StartupTemperature=dT.

This simply means the curve was set at a temperature 1.80° C. less than the temperature of the current test run. The reflectivity of the sample is measured, and this is converted into a concentration of analyte in the sample (in mg/dL, for example). This formula applies the correction factor from the chip to calculate the corrected value:

K=UncorrectedK+(dT*(long)wCorrectionFactor/100).

This is an example of an algorithm that may be applied. Similar algorithms may be used for temperature corrections in lipids and corrections for hematocrits. In the case of hematocrits, instead of measuring a starting temperature, a standard hematocrit level is assumed and calculated from that standard value based on a measured and calculated hematocrit value.

While specific embodiments have been described in detail in the foregoing detailed description and illustrated in the accompanying FIGURE, it will be appreciated by those skilled in the art that various modifications and alternatives to those details could be developed in light of the overall teachings of the disclosure and the broad inventive concepts thereof. It is understood, therefore, that the scope of this disclosure is not limited to the particular examples and implementations disclosed herein but is intended to cover modifications within the spirit and scope thereof as defined by the appended claims and any and all equivalents thereof. Note that, although particular embodiments are shown, features of each attachment may be interchanged between embodiments.

What is claimed as new and desired to be protected by Letters Patent of the United States is:

1. A system for detection of an analyte, comprising:
a lateral flow test strip, including a stripe for reacting with an analyte; and
a meter including and executing instructions for reading the lateral flow test strip and determining a level of the analyte, wherein the level of the analyte determined is adjusted according to an ambient temperature.

2. The system of claim 1, wherein the instructions include an algorithm for correlating a characteristic of the lateral flow test strip to the level of the analyte.

3. The system of claim 2, wherein the characteristic is color.

4. The system of claim 3, wherein the meter includes a thermometer, the thermometer operable for detecting the ambient temperature.

5. The system of claim 1, wherein the analyte is an enzyme.

6. The system of claim 5, wherein the enzyme is A1C.

7. The system of claim 4, wherein the algorithm is based on a calibration for 25° C. and the characteristic is adjusted based on the difference in the ambient temperature from the calibration.

8. The system of claim 1, wherein the stripe includes antibodies for the analyte.

9. A chip for use with a lateral flow test strip, the lateral flow test strip including a stripe for reacting with an analyte, a meter including and executing instructions for reading the lateral flow test strip and determining a level of the analyte, the chip comprising:
a non-transitory computer medium readable by the meter, the non-transitory computer medium including instructions for determining the level of the analyte adjusted according to ambient temperature.

10. The chip of claim 9, wherein the instructions include an algorithm for correlating a characteristic of the lateral flow test strip to the level of the analyte.

11. The chip of claim 10, wherein the characteristic is color.

12. The chip of claim 11, wherein the meter includes a thermometer, the thermometer operable for detecting the ambient temperature.

13. The chip of claim 9, wherein the analyte is an enzyme.

14. A method of determining an analyte level, the method comprising:
providing a lateral flow test strip, including a stripe for reacting with an analyte; and a meter including and executing instructions for reading the lateral flow test strip and determining a level of the analyte;
placing a sample on the lateral flow test strip;
reading the lateral flow test strip with the meter; and
adjusting the level of the analyte determined by the meter based on the ambient temperature.

15. The method of claim 14, wherein the instructions include an algorithm for correlating a characteristic of the lateral flow test strip to the level of the analyte.

16. The method of claim 15, wherein the characteristic is color.

17. The method of claim 16, wherein the meter includes a thermometer, the thermometer operable for detecting the ambient temperature.

18. The method of claim 14, wherein the analyte is an enzyme.
